(19) United States
(12) Patent Application Publication
(10) Pub. No.: US 2011/0311599 A1
(43) Pub. Date: Dec. 22, 2011

(54) GRANULATED POWDER CONTAINING VEGETABLE PROTEINS AND FIBERS, PROCESS FOR PRODUCING SAME, AND USE THEREOF

(75) Inventors: Bernard Boursier, Violaines (FR); Damien Passe, Douai (FR)

(73) Assignee: ROQUETTE FRERES, Lestrem (FR)

(21) Appl. No.: 13/203,029

(22) PCT Filed: Feb. 25, 2010
(86) PCT No.: PCT/FR2010/050327

§ 371 Date: Aug. 24, 2011

(30) Foreign Application Priority Data
Mar. 2, 2009 (FR) 0951293

(51) Int. Cl. A61K 8/02; A01N 25/12; C09K 8/035; A23L 1/05; C11D 3/38; A23J 1/14; A23J 3/14; A61K 9/14; C09K 3/00

(52) U.S. Cl. 424/401; 424/400; 507/104; 252/182.12; 510/108; 426/640; 426/573

(57) ABSTRACT

The present invention concerns a granulated powder containing at least one vegetable protein and at least one vegetable fiber, characterized in that it has a laser volume mean diameter D4,3 of between 10 μm and 500 μm, preferably between 50 μm and 350 μm, and even more preferably between 70 μm and 250 μm, and a dry matter content, determined after stoving at 130° C. for 2 hours, of greater than 80%, preferably greater than 85%, and even more preferably greater than 90%. The present invention also concerns a process for manufacturing this granulated powder as well as its use in various industrial field, and more particularly in the food-processing field, where it is used as a functional agent such as an emulsifying, overrun, stabilizing, thickening and/or gelling agent, in particular for totally or partially replacing certain animal proteins in the preparation of food products.

### FIELD OF THE INVENTION

The subject of the present invention is a granulated powder containing vegetable proteins and fibers, and also the process for producing same and the uses thereof.

### TECHNICAL BACKGROUND

Dietary habits have altered profoundly in industrialized countries since the Second World War and even more recently driven by the food-processing industry, the increasing influence of which on the nutritional behavior of populations tends to gradually blur the differences related to the conventional nutritional habits. This change probably contributes to increasing the risks of lithiasis, cardiovascular risks, and the risks of diabetes, obesity and certain cancers of nutritional origin in industrial societies where the daily energy needs have a tendency to become reduced in an increasing number of individuals with increasingly sedentary lifestyles.

Proteins represent, after carbohydrates and lipids, the third major energy source in our diet. They are provided both by products of animal origin (meats, fish, eggs, dairy products) and by plant foods (cereals, legumes, etc.). Daily protein needs are between 12% and 20% of the food intake. In industrialized countries, these intakes are predominantly in the form of proteins of animal origin. Studies show that we are consuming too many proteins of animal origin (70% of our intakes on average) and not enough vegetable proteins (30%). In addition, our food is too high in lipids, in particular in saturated fatty acids, and in sugars, and too low in fibers. In terms of protein intake, insufficiency like excess is prejudicial: in the event of insufficient intake, there is a risk of development and growth being disturbed. In the event of excessive intake, the amino acids constituting the proteins are oxidized or converted to carbohydrates or to fats. Such an excess is perhaps not without unfavorable consequences, especially in the case of animal proteins: in addition to the actual risk of oxidation and conversion of amino acids, it should be remembered that foods high in animal proteins are often also high in lipids and in saturated fatty acids. A recent study implicates the responsibility of excess animal proteins in the generation of subsequent obesity.

In addition, the advantages for the health are obvious since excessive consumption of animal proteins has been brought to the fore in causes for the increase in certain cancers and cardiovascular diseases.

In addition, intensive farming of animals generates serious environmental problems. Meat production requires twice as much water and two to four times more space than the production necessary for a plant-based diet. Animal farming also represents considerable soil and air pollution. It was recently proved that pollution from cattle farming exceeded motor vehicle pollution in terms of nitrogen waste.

Finally, animal farming represents a formidable waste of the world's water resources: 7 kg of cereals are necessary to produce 1 kg of beef-4 kg for producing 1 kg of pork-2 kg for producing 1 kg of poultry. Farm animals are fed with cereals that are edible for humans, such as soybean (the term then used is cake) and corn. In Brazil, soybean is today the main cause of deforestation of the Amazonia.

Thus, animal proteins derived from meat have many disadvantages, both in terms of health and in terms of environment.

In parallel, animal proteins derived from milk or from eggs can be allergenic, leading to reactions which are very bothersome, or even dangerous, in everyday life.

Thus, eggs are food allergens (a type of allergen) which penetrate via the digestive tract and which, in certain individuals, can cause a release of histamine by the cells of the organism. It is this substance which is responsible for the symptoms of inflammation and which leads to contraction of the bronchial muscles. Hypersensitivity is most commonly related to the egg white. On the other hand, in some individuals it is the proteins contained in the yolk which cause allergic reactions. Egg allergy is particular since it causes the entire range of symptoms associated with food allergies, such as bloating, digestive problems, skin rashes, nausea, diarrhea, asthma attacks and eczema. Egg white allergy can go as far as anaphylactic shock, a violent reaction which can lead to the death of the allergic individual if the latter does not immediately receive an injection of adrenalin.

Dairy product allergy is one of the most widespread allergic reactions. Studies demonstrate that 65% of individuals who suffer from food allergies are allergic to milk. The adult form of milk allergy, herein referred to as “dairy products allergy”, is a reaction of the immune system which creates antibodies in order to combat the unwanted food. This allergy is different than cow's milk protein (bovine protein) allergy, which affects newborns and infants. Dairy product allergy causes varied symptoms, such as constipation, diarrhea, flatulence, eczema, urticaria, nausea, migraines, infections, abdominal cramps, nasal congestion and even serious asthma attacks. Allergic individuals should completely eliminate milk, dairy products and derivatives thereof from their diet. The following terms are indicators of the presence of cow's milk or derivatives thereof in the ingredients of a product: buttermilk, calcium caseinate, sodium caseinate, casein, caseinate, hydrolyzed casein, dried milk solids, lactalbumin, lactose, lactoglobulin, low-fat milk, milk powder, condensed milk and whey.

Another major problem associated with milk proteins is their cost, which never ceases to increase. The application of milk quotas has caused, on the one hand, a drastic reduction in the amount of milk proteins available for the production of food products and, on the other hand, large fluctuations in their price. Manufacturers are increasingly seeking substitute products for these milk proteins.

In view of all the disadvantages, whether they are economical, environmental or nutritional, associated with the consumption of animal proteins derived from meat and/or derived products, there is, as a result, great interest in the use of substitute proteins, also called alternative proteins, classified among which are vegetable proteins. The alternative market for these proteins is developing rapidly, for many reasons. These proteins have a profound influence on the formulation of balanced foods and diets based on a low glycemic index (GI) and a high protein intake, and conventional manufacturers are beginning to seek new sources of proteins in order to enrich their products.

For example, document WO 2008/066308 describes a food composition containing an optimum combination of nutrients essential for a balanced diet, combined with soybean proteins. This composition makes it possible to reduce the problems of obesity by reducing, inter alia, harmful protein intakes.

Document EP 0522800 describes a novel method for treating a vegetable protein concentrate to enhance its functionality for binding fat and water and also its use as a replacement for animal proteins in the manufacture of sausages.

Document EP 0238946 describes an improved protein isolate derived from seeds of a grain legume with a relatively low lipid content, the method for preparing same and also the use thereof as an additive in the manufacture of sausages and saveloys.

The applicant company also focused in on this research in order to be able to meet the increasing demands from manufacturers for compounds having advantageous functional properties without, however, having the drawbacks of certain already-existing compounds.

Specifically in fields as diversified as nutrition, pharmacy, cosmetics, agrochemistry, construction materials and paper-cardboards, manufacturers are constantly searching for new compounds which have a positive and beneficial image in terms of health and which are capable of modifying the functional properties of media in order to manufacture products having varied textures.

Thus, the applicant has carried out considerable research studies on Vegetable Protein Materials (VPM) as food ingredients. This interest in VPM is first of all due to their numerous functional properties, but also to advantageous nutritional qualities by virtue of their “essential” amino acid composition.

In the present application, the term “VPM” denotes food ingredients obtained from oleaginous plants, leguminous plants or cereals by reduction or elimination of some of the main non-protein constituents (water, oil, starch, other carbohydrates), so as to obtain a protein content (N×6.25) of 50% or more. The protein content is calculated on the basis of the dry weight excluding the vitamins and mineral salts.

VPM are increasingly used in food applications. They have become an important ingredient owing to their overrun, texturing, emulsifying, thickening, stabilizing, foaming or gelling properties, which are constantly being improved, for use in known applications or else quite simply in completely new creations.

One of the objects of the present invention is therefore to propose vegetable proteins as a replacement for animal proteins, while at the same time making it possible to retain, in a product in which they are used, functional properties, a flavor and palatability and also a nutritional value which are at least similar, or even improved. The product will have an equivalent nutritional value:
if its protein quality is not inferior to that of the product of origin, and
if it contains an amount of proteins (N×6.25), mineral salts and vitamins equivalent to that present in the products of animal origin.

Proteins play a major role in the organoleptic quality of many fresh or manufactured foods, for instance the consistency and the texture of meat and meat products, of milk and derivatives, of pasta and of bread. These food qualities very frequently depend on the structure and the physicochemical properties of the protein components or quite simply on their functional properties.

In the present application, the term “functional properties of food ingredients” means any non-nutritional property which influences the usefulness of an ingredient in a food. These various properties would contribute to obtaining the desired final characteristics of the food. Some of these functional properties are solubility, hydration, viscosity, coagulation, stabilization, texturing, dough formation, and foaming and coagulating properties.

In addition to the substitution of animal proteins and, as a result, the elimination of many of the disadvantages associated with their use, the applicant company has also concentrated on the formation of novel ready-to-use food ingredients, containing, in addition to the VPM, other compounds having different but complementary functional and/or nutritional properties.

Indeed, nowadays, in the interests of maximum cost-effectiveness, there is an increasing desire to simplify manufacturing processes on the part of manufacturers, and most particularly in the food-processing industry.

This simplifying of food product manufacturing processes results in particular in a reduction in the number of compounds used, and in particular in the ingredients involved in preparing the final products. This reduction in ingredients makes it possible simultaneously to limit the manufacturing times of the products, to simplify the manufacturing processes and to reduce the costs thereof. However, it must not alter the texture or any of the functional, nutritional, sensory or organoleptic properties of said products.

Still with a desire to simplify food product manufacturing processes, manufacturers are also increasingly demanding with respect to the form of said ingredients used. The dry form is by far the form preferred by manufacturers, whether in terms of preservation, storage or handling, compared with a liquid form for example, which is much less stable over time. Nevertheless, the use of ingredients in pulverulent form has the disadvantage that these products are sometimes difficult to dissolve, which can lead to settling out, and poor dispersibility with the formation of lumps and therefore uneven distribution of the ingredients during the process. What is more, the handling of pulverulent products poses safety problems due, inter alia, to the dry residues that handlers may breathe in, with, in addition, risks of fire and explosion.

As a result of all the above, there is a real, unmet need to have a composition used as a substitute for proteins of animal origin, which has several advantageous functional properties enabling it to reduce the number of additives used in the manufacture of a finished product while at the same time providing it with technological characteristics similar to those obtained by using said additives separately, and which is in a dry but nonpulverulent form which can be easily hydrated.

Armed with this observation and after a considerable amount of research, the applicant company has, to its credit, reconciled all these objectives reputed up until now to be difficult to reconcile, by proposing a novel composition containing, inter alia, vegetable proteins, characterized in that it:
combines a vegetable protein and a vegetable fiber, itself having an advantageous and desired functional characteristic and/or nutritional characteristic and/or technological characteristic,
is in dry but nonpulverulent form, i.e. in a granular form; it is referred to as a granulated powder,
has a dry matter content of greater than 80%, preferably greater than 85%, and even more preferably greater than 90%,
has an “instant” nature, i.e. this granulated powder has very good wettability, dispersability and solubility in water.

Said granulated powder is characterized in that it exhibits, compared with the simple physical mixtures of powder described in the prior art, better dispersion in water and better dissolution under cold conditions, and better flowability for metering operations, and in that it offers a better environment for handling the powders owing to the absence of dust. What is more, this granulated powder has improved functional characteristics, that the simple physical mixing of the various constituents would not have made it possible to obtain.

### SUMMARY OF THE INVENTION

The subject of the present invention is therefore a granulated powder comprising at least one protein of vegetable origin and at least one fiber of vegetable origin, characterized in that it has a laser volume mean diameter D4,3 of between 10 μm and 500 μm, preferably between 50 μm and 350 μm, and even more preferably between 70 μm and 250 μm, and a dry matter content, determined after stoving at 130° C. for 2 hours, of greater than 80%, preferably greater than 85%, and even more preferably greater than 90%.

The present invention also relates to the process for obtaining this granulated powder and to the use thereof in various industrial fields, and more particularly in the food-processing field, where it is used as a functional agent such as an emulsifying, overrun, stabilizing, thickening and/or gelling agent, in particular for totally or partially replacing certain animal proteins in the preparation of food products.

### DETAILED DESCRIPTION OF EMBODIMENTS

The present invention relates to a granulated powder comprising at least one vegetable protein and at least one vegetable fiber, characterized in that it has a laser volume mean diameter D4,3 of between 10 μm and 500 μm, preferably between 50 μm and 350 μm, and even more preferably between 70 μm and 250 μm, and a dry matter content, determined after stoving at 130° C. for 2 hours, of greater than 80%, preferably greater than 85%, and even more preferably greater than 90%.

In the present invention, said granulated powder is characterized in that the weight ratio of the vegetable protein to the vegetable fiber is between 99:1 and 1:99, preferably between 80:20 and 20:80, even more preferably between 65:35 and 35:65, and in particular between 55:45 and 45:55.

In the present invention, said granulated powder is characterized in that the sum of the amounts of vegetable protein and of vegetable fiber is between 30% and 100%, and preferably between 50% and 100%, of the total mass of said granulated powder (dry/dry).

In the present invention, the term “vegetable protein” denotes all proteins derived from cereals, oleaginous plants, leguminous plants and tuberous plants.

In the present invention, the term “vegetable protein” also denotes all proteins derived from algae and from microalgae.

These vegetable proteins can be used alone or as mixtures, chosen from the same family or from different families.

Thus, said granulated powder according to the invention is characterized in that the vegetable protein is a protein derived from the family of cereals, oleaginous plants, leguminous plants, tuberous plants, algae and microalgae, used alone or as a mixture, chosen from the same family or from different families.

In the present invention, the terms “algae” and “microalgae” are intended to mean eukaryotic organisms devoid of roots, stalks and leaves, but having chlorophyll and also other pigments that are incidental to oxygen-producing photosynthesis. They are blue, red, yellow, golden and brown, or else green. They represent more than 90% of marine plants and 18% of the vegetable kingdom, and comprise 40 000 to 45 000 species. Algae are organisms that are extremely varied both in terms of their size and their shape and in terms of their cell structure. They live in an aquatic or very humid environment. They contain many vitamins and trace elements, and are true concentrates of active agents that are stimulants of and beneficial to health and beauty. They have anti-inflammatory, hydrating, soothing, regenerating, firming and anti-aging properties. They also have “technological” characteristics which make it possible to give a food product texture. Specifically, the much-vaunted additives E400 to E407 are in fact merely compounds extracted from algae, the thickening, gelling, emulsifying and stabilizing properties of which are used.

Microalgae in the strict sense are undifferentiated unicellular or multicellular microscopic algae; they are photosynthetic microorganisms separated into two polyphyletic groups: eukaryotes and prokaryotes. They live in strongly aqueous environments, and can have flagellar mobility.

According to one preferred embodiment, the microalgae are chosen from the group consisting of Chlorella, Spirulina and Odontella.

According to an even more preferred embodiment, the microalgae of the present invention are derived from the Chlorella genus, and preferably from Chlorella vulgaris, Chlorella pyrenoidosa, Chlorella regularis or Chlorella sorokiniana, and even more preferably from Chlorella vulgaris.

In the present application, the term “cereals” is intended to mean cultivated plants of the grass family producing edible seeds, for instance wheat, oats, rye, barley, corn, sunflower, sorghum or rice. The cereals are often milled in the form of flour, but are also as grains and sometimes in whole-plant form (fodders).

In the present application, the term “tuberous plants” is intended to mean all the storage organs, which are generally underground, which ensure plant survival during the winter season and often plant multiplication by the vegetative process. These organs are bulging owing to the accumulation of storage substances. The organs transformed into tubers may be:
the root: carrot, parsnip, cassava, konjac,
the rhizome: potato, Jerusalem artichoke, Japanese artichoke, sweet potato,
the base of the stalk (more specifically the hypocotyl): kohlrabi, celeriac,
the root+hypocotyl combination: beetroot, radish.

In the present application, the term “oleaginous plants” denotes plants cultivated specifically for their seeds or their fruits rich in fats, from which oil for dietary, energy or industrial use is extracted, for instance rapeseed, groundnut, sunflower, soybean, sesame and the castor oil plant.

For the purpose of the present invention, the term “leguminous plants” is intended to mean any plants belonging to the family Caesalpiniaceae, the family Mimosaceae or the family Papilionaceae, and in particular any plants belonging to the family Papilionaceae, for instance pea, bean, broad bean, horse bean, lentil, alfalfa, clover or lupine.

This definition includes in particular all the plants described in any one of the tables contained in the article by R. Hoover et al., 1991 (Hoover R. (1991) “Composition, structure, functionality and chemical modification of legume starches: a review” Can. Jr. Physiol. Pharmacol., 69 pp. 79-92).

According to one preferred embodiment of the present invention, the vegetable protein belongs to the leguminous plants proteins.

According to another preferred embodiment, the leguminous plants protein is chosen from the group comprising pea, bean, broad bean and horse bean, and mixtures thereof. According to another preferred embodiment, the leguminous plants protein is chosen from the group comprising alfalfa, clover, lupine, pea, bean, broad bean, horse bean and lentil, and mixtures thereof, and preferably from pea, bean, broad bean and horse bean, and mixtures thereof.

Even more preferably, said leguminous plant protein is pea.

The term “pea” is here considered in its broadest sense, and includes in particular:
all wild-type varieties of smooth pea and of wrinkled pea, and
all mutant varieties of smooth pea and of wrinkled pea, irrespective of the uses for which said varieties are generally intended (food for human consumption, animal feed and/or other uses).

Said mutant varieties are in particular those known as “r mutants”, “rb mutants”, “rug 3 mutants”, “rug 4 mutants”, “rug 5 mutants” and “lam mutants” as described in the article by C-L Heydley et al., entitled “Developing novel pea starches” Proceedings of the Symposium of the Industrial Biochemistry and Biotechnology Group of the Biochemical Society, 1996, pp. 77-87.

Even more preferably, said leguminous plant protein is smooth pea.

Indeed, pea is the leguminous plant with protein-rich seeds which, since the 1970s, has been most widely developed in Europe and mainly in France, not only as a protein source for animal feed, but also for foods for human diet.

The pea proteins are, like all legume proteins, made up of three main classes of proteins: globulins, albumins and “insoluble” proteins.

The value of pea proteins lies in their good emulsifying capacities, their lack of allergenicity and their low cost, which makes an economical functional ingredient.

Furthermore, the pea proteins contribute favorably to sustainable development and their carbon impact is very positive. This is because the pea cultivation is environmentally friendly and does not require nitrogenous fertilizers, since pea fixes nitrogen from the air.

Besides, in native globular form, pea proteins are water-soluble, which makes it possible to envision incorporating them into emulsions.

According to the present invention, the term “pea protein” preferably denotes the pea proteins which are mainly in native globular form, globulins, or albumins.

Even more preferably, the pea proteins used according to the invention are in the form of a composition of pea protein having:
a total protein content (N×6.25), expressed in grams of dry product, of at least 60% by weight of dry product. Preferably, in the context of the present invention, use is made of a protein composition having a high protein content of between 70% and 97% by weight of dry product, preferably between 76% and 95%, even more preferably between 78% and 88%, and in particular between 78% and 85%,
a soluble protein content, expressed according to a test for measuring the water-solubility of proteins, of between 20% and 99%. Preferably, in the context of the present invention, use is made of a protein having a high soluble protein content of between 45% and 90%, even more preferably between 50% and 80%, and in particular between 55% and 75%.

In order to measure the total protein content, the soluble nitrogenous fraction contained in the sample can be quantitatively determined according to the Kjeldahl method, and then the total protein content is obtained by multiplying the nitrogen content, expressed as percentage weight of dry product, by the factor 6.25. This method is well known to those skilled in the art.

In the present invention, the total protein content can also be measured by quantitatively determining the soluble nitrogenous fraction contained in the sample according to the method of A. Dumas, 1831, Annales de chimie [Annals of chemistry], 33, 342, as cited by Buckee, 1994, in Journal of the Institute of Brewing, 100, pp. 57-64, and then the total protein content is obtained by multiplying the nitrogen content, expressed as percentage weight of dry product, by the factor 6.25. This method, also known as the combustion method for determining nitrogen, consists of total combustion of the organic matrix under oxygen. The gases produced are reduced by copper and then dried, and the carbon dioxide is trapped. The nitrogen is then quantified using a universal detector. This method is well known to those skilled in the art.

To determine the soluble protein content, the content of proteins soluble in water of which the pH is adjusted to 7.5+/−0.1 using a solution of HCl or NaOH is measured by means of a method of dispersion of a test specimen of the sample in distilled water, centrifugation and analysis of the supernatant. 200.0 g of distilled water at 20° C.+/−2° C. are placed in a 400 ml beaker, and the whole is stirred magnetically (magnetic bar and rotation at 200 rpm). Exactly 5 g of the sample to be analyzed are added. The mixture is stirred for 30 min, and centrifuged for 15 min at 4000 rpm. The method for determining nitrogen is carried out on the supernatant according to the method previously described.

These vegetable protein, and in particular pea protein, compositions preferably contain more than 50%, 60%, 70%, 80% or 90% of proteins of more than 1000 Da. In addition, these vegetable protein, and in particular pea protein, compositions preferably have a molecular weight distribution profile consisting of:
1% to 8%, preferably from 1.5% to 4%, and even more preferably from 1.5% to 3% of proteins of more than 100 000 Da,
20% to 55%, preferably from 25% to 55% of proteins of more than 15 000 and of at most 100 000 Da,
15% to 30% of proteins of more than 5000 and of at most 15 000 Da,
and from 25% to 55%, preferably from 25% to 50%, and even more preferably from 25% to 45% of proteins of at most 5000 Da.

The determination of the molecular weights of the constitutive proteins of said pea protein compositions is carried out by size exclusion chromatography under denaturing conditions (SDS+2-mercaptoethanol); the separation is carried out according to the size of the molecules to be separated, the molecules of large size being eluted first.

Examples of pea protein compositions according to the invention, and also the details of the method for determining the molecular weights, can be found in patent WO 2007/017572, of which the applicant company is also the proprietor.

According to the present invention, said vegetable proteins, and in particular pea proteins, used for producing the granulated powder can also be “vegetable protein concentrates” or “vegetable protein isolates”, preferably “pea protein concentrates” or “pea protein isolates”. The vegetable protein, and in particular pea protein, concentrates and isolates are defined from the viewpoint of their protein content (cf. the review by J. Gueguen from 1983 in Proceedings of European congress on plant proteins for human food (3-4) pp 267-304)
the vegetable protein, and in particular pea protein, concentrates are described as having a total protein content of from 60% to 75% on a dry basis, and
the vegetable protein, and in particular pea protein, isolates are described as having a total protein content of 90% to 95% on a dry basis,
   the protein contents being measured by the Kjeldahl method (cf. above), the nitrogen content being multiplied by the factor 6.25.

In another embodiment of the present invention, the vegetable protein, and in particular pea protein, compositions that can be used may also be “vegetable protein hydrolyzates”, preferably “pea protein hydrolyzates”. The vegetable protein, and in particular pea protein, hydrolyzates are defined as preparations obtained by enzyme hydrolysis or chemical hydrolysis, or by both simultaneously or successively, of vegetable proteins, and in particular pea proteins. The protein hydrolyzates are composed of a mixture of peptides of various sizes and of free amino acids. This hydrolysis can have an impact on the solubility of the proteins. The enzyme and/or chemical hydrolysis is, for example, described in patent application WO 2008/001183. Preferably, the protein hydrolysis is not complete, i.e. does not result in a composition comprising only or essentially amino acids and small peptides (from 2 to 4 amino acids). Thus, the hydrolyzates according to the invention are not HPV compositions. The preferred hydrolyzates comprise more than 50%, 60%, 70%, 80% or 90% of proteins of more than 500 Da.

The processes for preparing protein hydrolyzates are well known to those skilled in the art and can, for example, comprise the following steps: dispersion of the proteins in water so as to obtain a suspension, hydrolysis of this suspension by means of the chosen treatment. Most commonly, it will be an enzymatic treatment combining a mixture of various proteases, optionally followed by a thermal treatment intended to inactivate the enzymes that are still active. The solution obtained can then be filtered through one or more membranes so as to separate the insoluble compounds, optionally the residual enzyme, and the high-molecular-weight peptides (greater than 10 000 daltons).

In one preferred embodiment, the vegetable proteins included in the granulated powder are gluten free. This embodiment is advantageous since there are a certain number of individuals who are gluten-intolerant.

Gluten is a group of proteins present in cereals, particularly in wheat, but also in rye, barley and oats. For most individuals, gluten is a normal protein which is readily digested by means of the stomach. However, a small section of the population is incapable of digesting gluten. These gluten-intolerant individuals are most generally denoted as suffering from celiac disease (also known as psilosis, gluten-intolerant enteropathy or gluten-sensitive enteropathy). This disease appears when there is a chronic reaction against certain protein chains present in some cereals. This reaction brings about the destruction of the intestinal villi of the small intestine, which causes malabsorption of nutrients and other more or less serious disorders. It is a very restricting disease for which, at the current time, there is unfortunately no curative treatment. According to the present invention, the granulated powder comprises at least one vegetable protein and at least one vegetable fiber.

In the present invention, the term “vegetable fiber” denotes soluble and/or insoluble vegetable dietary fibers. Said fibers denote not only fibrous matter in the strict sense, but also an entire series of different compounds which are contained almost exclusively in foods of vegetable origin and which have the common property that they cannot be broken down by the digestive enzymes of human beings. Almost all dietary fibers are carbohydrate polymers. Over the last few years, nutritionists have focused on a new type of dietary fibers: resistant starch. It is a starch or starch fraction which is not digested in the small intestine and which is fermented by the bacteria of the colon.

Unlike conventional vegetable fibers, these starches have the advantage of not modifying the appearance of the product into which they are incorporated, and in a way constitute a source of fibers invisible to the naked eye. These starches are recommended in many applications.

Thus, in the present invention, the vegetable fiber is chosen from soluble fibers, insoluble fibers and any mixtures thereof.

According to one advantageous embodiment of the present invention, the granulated powder comprises at least one vegetable protein and at least one soluble vegetable fiber.

According to one preferred embodiment of the present invention, the granulated powder comprises pea proteins and at least one soluble vegetable fiber.

Preferably, said soluble fiber of vegetable origin is chosen from the group consisting of fructans, including fructooligosaccharides (FOSs) and inulin, glucooligosaccharides (GOSs), isomaltooligosaccharides (IMOs), trans-galactooligosaccharides (TOSs), pyrodextrins, polydextrose, branched maltodextrins, indigestible dextrins and soluble oligosaccharides derived from oleaginous plants or protein-producing plants.

The term “soluble fiber” is intended to mean fibers soluble in water. The fibers can be assayed according to various AOAC methods. By way of example, mention may be made of AOAC methods 997.08 and 999.03 for fructans, FOSs and inulin, AOAC method 2000.11 for polydextrose, AOAC method 2001.03 for assaying the fibers contained in branched maltodextrins and indigestible dextrins, or AOAC method 2001.02 for GOSs and also soluble oligosaccharides derived from oleaginous plants or protein-producing plants. Among the soluble oligosaccharides derived from oleaginous plants or protein-producing plants, mention may be made of soya, rapeseed or pea oligosaccharides.

According to one particularly advantageous embodiment of the present invention, the granulated powder comprises pea proteins associated with soluble vegetable fibers which are branched maltodextrins.

The term “branched maltodextrins” is intended to mean the specific maltodextrins identical to those described in patent EP 1 006 128-B1 of which the applicant is the proprietor. These branched maltodextrins have the advantage of representing a source of indigestible fibers beneficial to the metabolism and to the intestinal equilibrium. In particular, use may be made of branched maltodextrins having between 15% and 35% of 1-6 glucosidic linkages, a reducing sugar content of less than 20%, a weight-average molecular mass MW of between 4000 and 6000 g/mol and a number-average molecular mass Mn of between 250 and 4500 g/mol.

Certain subfamilies of branched maltodextrins described in the abovementioned application can also be used in accordance with the invention. They are, for example, high-molecular-weight branched maltodextrins having a reducing sugar content at most equal to 5 and an Mn of between 2000 and 4500 g/mol. Low-molecular-weight branched maltodextrins having a reducing sugar content of between 5% and 20% and a molecular weight Mn of less than 2000 g/mol can also be used.

In the present application, the pyrodextrins denote the products obtained by heating starch brought to a low moisture content, in the presence of acid or basic catalysts, and which generally have a molecular weight of between 1000 and 6000 daltons. This dry roasting of the starch, most commonly in the presence of acid, leads to both depolymerization of the starch and rearrangement of the starch fragments obtained, resulting in highly branched molecules being obtained. This definition targets in particular the “indigestible” dextrins, having an average molecular weight of about 2000 daltons.

Polydextrose is a soluble fiber produced by thermal polymerization of dextrose, in the presence of sorbitol and of an acid as catalyst. An example of such a product is, for example, Litesse® sold by Danisco.

An example of a combination with a particularly advantageous vegetable protein is the use of Nutriose®, which is an full range of soluble fibers, recognized for their benefits, and produced and sold by the applicant. The products of the Nutriose® range are partially hydrolyzed wheat starch or corn starch derivatives which contain up to 85% fibers. This richness in fiber makes it possible to increase the digestive tolerance, to improve calorie control, to prolong energy release and to obtain a low sugar content. In addition, the Nutriose® range is one of the most well tolerated fibers available on the market. It shows higher digestive tolerance, allowing better incorporation than other fibers, thereby representing real dietary advantages.

According to one advantageous embodiment of the invention, the granulated powder contains vegetable proteins combined with insoluble vegetable fibers.

According to another preferred embodiment of the present invention, the granulated powder comprises pea proteins and at least one insoluble vegetable fiber.

Preferably, said insoluble vegetable fiber is chosen from the group consisting of resistant starches, cereal fibers, fruit fibers, fibers from vegetables, leguminous plants fibers and mixtures thereof.

Mention may, for example, be made of fibers such as bamboo, pea or carrot fibers.

According to a first variant, said powder comprises pea proteins and at least one insoluble vegetable fiber, and preferably one leguminous plant fiber and even more preferably one pea fiber.

According to a second variant, the insoluble vegetable fiber is a resistant starch. Natural resistant starches or resistant starches obtained by chemical and/or physical and/or enzymatic modification may be used without distinction.

According to the present invention, the term “resistant starch” denotes a starch or a starch fraction which is not digested in the small intestine and which is fermented by the bacteria of the colon. Four categories of resistant starch have been identified:
encapsulated starches, present in most unrefined vegetable foods such as dry vegetables, said starches being inaccessible to enzymes (RS1),
the granular starch of certain raw foods, such as bananas or potatoes, and amylose-rich starches (RS2),
retrograded starches, which are found in foods which have been cooked and then refrigerated or frozen (RS3),
chemically modified starches such as, in particular, etherified or esterified starches (RS4).

The resistant starches proposed, in particular, by the company National Starch, such as those sold under the name Hi-Maize®, are derived from corn varieties rich in amylose and behave like insoluble fibers. RS3-type resistant starches are also proposed under the name Novelose®.

These resistant starches reduce the glycemic response, improve the health of the digestive system by virtue of their prebiotic properties and contribute to the regularity of transit, without having a high calorie content.

According to a third variant, the insoluble vegetable fiber comprises a mixture of at least one resistant starch and of a pea fiber.

Preferably, a resistant starch derived from starch having an amylose content of greater than 50% will be used. The Eurylon® amylose-rich starches sold by the applicant are particularly suitable.

According to another particularly advantageous embodiment of the invention, the granulated powder comprises pea proteins and a mixture of soluble and insoluble fibers.

Advantageously, the soluble fibers are branched maltodextrins when the insoluble fibers are chosen from leguminous plants fibers and resistant starches, or are a mixture of the two.

According to one particularly advantageous feature of the invention, said leguminous plant from which the leguminous plants fibers or the leguminous plants proteins are derived is selected from the group comprising alfalfa, clover, lupine, pea, bean, broad bean, horse bean, lentil and mixtures thereof. Thus, the invention relates in particular to a granulated powder comprising proteins and fibers derived from a leguminous plant selected from the group comprising alfalfa, clover, lupine, pea, bean, broad bean, horse bean, lentil, and mixtures thereof, preferably derived from pea.

In the context of the present invention, the expression “granulated powder” signifies that there is intimate mixing between the various components of this powder, that their distribution within the powder is substantially homogeneous, and that they are not only linked to one another by simple physical mixing. Interactions between the constituents can occur both outside the particle and inside. In particular, each grain of said granulated powder comprises both vegetable proteins and vegetable fibers.

In one particular embodiment, the granulated powder is not coated.

In another particular embodiment, the granulated powder does not comprise gluten.

Conversely, in the present invention, the expression “simple mixing” signifies that there is no intimate mixing between the various constituents, and that there only has been simple physical mixing by contact. There is no interaction between the constituents since they are virtually not in contact with one another. In particular, in a simple mixture, some particles of the powder will consist of vegetable proteins, whereas other particles of said powder will consist of vegetable fibers.

Indeed, in order to produce said granulated powder, the applicant company has noted that it is advisable to use a mixture of at least one vegetable protein and at least one vegetable fiber, and to modify its physical characteristics by employing a suitable process, such that very advantageous functional properties, which cannot be obtained if each compound is used separately or if the compounds are used simultaneously but in the form of a simple mixture of powders, are simultaneously obtained.

In the present invention, said granulated powder is prepared by means of a drying process according to a technique chosen from the group consisting of spray-drying, granulation, extrusion or any other drying means known to those skilled in the art, and under conditions suitable for the chosen equipment, capable of enabling the production of a granulated powder according to the invention.

Thus, the present invention is also directed toward a process for manufacturing the abovementioned granulated powder. Said manufacturing process consists in drying conjointly at least two constituents, and comprises a step of bringing at least one vegetable protein into intimate contact with at least one vegetable fiber, it being possible for this step of bringing into intimate contact to be carried out according to any process known to those skilled in the art, and in particular according to a technique chosen from spray-drying, granulation and extrusion, and any combination of at least two of these techniques, such that said step of bringing into intimate contact results in a dry matter content, determined after stoving at 130° C. for 2 hours, of greater than 80%, preferably greater than 85%, and even more preferably greater than 90%. By way of example, mention will be made of a process for manufacturing said granulated powder according to a single spray-drying technique, or according to a single granulation technique, or else according to a combination of a spray-drying technique followed by a granulation technique.

Thus, according to a first variant of the invention, said granulated powder can be produced according to a manufacturing process which comprises a step of spray-drying a suspension of at least one vegetable protein and of at least one vegetable fiber, said spray-drying step being followed by a step of granulation of the “spray-dried” powder on a granulator. According to this first variant, a suspension to be spray-dried is prepared, containing at least one protein of vegetable origin, preferably a pea protein, and at least one vegetable fiber, and preferably a branched maltodextrin, in the required proportions. Still according to this variant, it is also possible to envision preparing one aqueous suspension to be spray-dried per constituent.

Still according to this variant, the suspension to be spray-dried can be prepared either from a dry composition of vegetable proteins, and preferably from a dry composition of pea proteins, i.e. in the form of a powder which is then diluted in water, or from a floc of vegetable proteins, and preferably of pea proteins. In this second alternative, the floc of vegetable proteins, and preferably the floc of pea proteins, is obtained by milling the vegetable flour, and preferably the pea flour, resuspending this milled flour in water, and then fractionating said suspension by any means known, moreover, to those skilled in the art, so as to isolate a protein-rich fraction. The proteins are then isolated from this fraction by means of a technique chosen from the group of techniques for precipitating proteins at their isoelectric pH and ultrafiltration-type membrane separation techniques. Finally, the separation of the precipitate (also referred to as “floc”) containing the soluble proteins is carried out on a centrifugal decanter or on a plate separator. The floc can be used as it is or suspended, depending on its dry matter content.

The spray-drying step is a unit drying operation which consists in converting into a powder a liquid, sprayed in the form of droplets brought into contact with a hot gas. This operation determines the size of the droplets produced (and their size grading), their path, their speed and, consequently, the final dimension of the dry particles, as well as the properties of the powders produced: flow, instant nature related to their solubility, density, compressibility, friability, etc.

The spray-drying step can be carried out in a spray dryer or a spray-drying tower, in which said suspension (or the suspensions) to be dried is (or are) divided in a stream of hot gas which provides the heat necessary for evaporating the solvent and absorbs, in order to evacuate it, the moisture released by the product during drying. The liquid mixture is introduced at the top via a nozzle or a turbine, and the “spray-dried” powder produced is harvested at the bottom of the tower. The dry solid is separated from the spray-drying gas by means of a cyclone (or cyclones), or by filtration (sleeve filter, for example). In certain cases, if this is found to be necessary, the tower can be filled with an inert gas in order to prevent oxidation phenomena.

The granulation step is carried out after the spray-drying step, and consists in spraying an aqueous solution onto the powder resulting from the spray-drying step. Such an operation, combining a spray-drying step followed by a granulation step, is conventionally carried out in a multi-effect spray dryer such as, for example, an MSD (multi-stage dryer) tower.

According to one preferred embodiment of this first variant, the process can be carried out according to the following steps:
1) preparing, at a temperature of between 15 and 70° C., and preferably between 15 and 50° C., a suspension of pea proteins and of branched maltodextrins, in which:
   said pea proteins have a soluble protein content of between 20% and 99%, preferably between 45% and 90%, even more preferably between 50% and 80%, and in particular between 55% and 75%;
   said branched maltodextrins have between 15% and 35% of 1-6 glucosidic linkages, a reducing sugar content of less than 20%, a molecular weight MW of between 4000 and 6000 g/mol and a number-average molecular mass Mn of between 250 and 4000 g/mol;
   the weight ratio of the pea proteins to the branched maltodextrins is between 99:1 and 1:99, preferably between 80:20 and 20:80, even more preferably between 65:35 and 35:65, and in particular between 55:45 and 45:55;
   the dry matter content of the suspension is between 25% and 50%, preferably between 30% and 40%;
1′) carrying out an optional first step of heat treatment at high temperature and for a short period of time in order to reduce the bacteriological risks of the suspension obtained according to 1, it being possible for said treatment to be chosen from HTST (high temperature short time) and UHT treatments;
1″) carrying out an optional second step of high-pressure homogenization of the suspension obtained according to 1), and independently of the optional first step;
2) maintaining said suspension of pea proteins and of branched maltodextrins at a temperature of between 15 and 80° C., and preferably between 15 and 50° C., or, in the event of step 1′) being carried out, bringing said suspension of pea proteins and of branched maltodextrins back to a temperature of between 15 and 80° C., and preferably between 15 and 50° C.;
3) spray-drying said suspension in an MSD-type spray-drying tower equipped with a high-pressure spray-drying nozzle with recycling of the fine particles at the top of the tower;
4) granulating in said spray-drying tower;
5) recovering the resulting granulated powder comprising the pea proteins and the branched maltodextrins.

As it will be exemplified hereinafter, the applicant company recommends using an MSD 20 tower sold by the company Niro.

The injection nozzle is chosen so as to obtain a pressure of between 50 and 300 bar, preferably about 150 bar, for a flow rate of between 100 and 150 l/h, preferably about 120 l/h.

The inlet air temperatures are set in the following way:
for the inlet air upstream of the top of the tower: temperature between 150 and 180° C., preferably 155° C.,
for the static fluidized bed: temperature between 50 and 120° C., preferably 84° C.,
for the vibrated fluidized bed: temperature of about 20° C.

The outlet temperature is then between 55 and 80° C., about 60° C.

The granulated powder according to the invention, containing cogranules, is finally recovered at the exit of the spray-drying tower.

According to a second variant of the invention, said granulated powder is produced according to a sole granulation process which makes it possible to carry out the step of bringing the various constituents into intimate contact. The granulation process can make use of two techniques well known to those skilled in the art: the dry granulation technique and the wet granulation technique.

According to one preferred embodiment of this second variant, the granulated powder is produced by wet granulation in a fluidized bed. An example of such a granulation is, for example, mentioned in patent EP 1 558 094, of which the applicant is the proprietor.

According to a third variant of the invention, said granulated powder is produced according to a single extrusion process. In this process, equipment comprising at least one extrusion die will be used, the temperature parameters being readily selected by those skilled in the art according to the water content of the composition before drying. The extruded composition is then successively subjected to cooling, milling and, optionally, sieving so as to result in the spray-dried powder according to the present invention.

The implementation of the drying processes described above, or processes for drying by any other drying means known to those skilled in the art, and under conditions suitable for the chosen equipment, produces a granulated powder composed of cogranules and containing the various starting compounds intimately linked to one another.

In one preferred embodiment of the invention, the process for preparing the granulated powder of the invention does not require the use of emulsifier, and in particular of lecithin or derivatives thereof or of mono- or diglycerides of dietary fatty acids (E471, E472c, etc.). Indeed the pea proteins have the advantage of exhibiting intrinsic emulsifying capacities which make it possible to dispense with said emulsifiers. Thus, the present invention relates in particular to a granulated powder according to the invention which does not comprise any emulsifier other than the pea proteins, and preferably which does not comprise lecithin or derivatives thereof.

The average size of the powder obtained in accordance with the invention can be characterized by its volume mean diameter (arithmetic mean) D4,3. It is between 10 μm and 500 μm, preferably between 50 μm and 350 μm, and even more preferably between 70 μm and 250 μm.

According to one preferred embodiment, the volume mean diameter D4,3 of said granulated powder is between 150 μm and 240 μm.

These values are determined on an LS 230 Laser diffraction particle size analyzer from the company Beckman-Coulter, equipped with its powder dispersion module (dry process), according to the technical manual and the specifications of the constructor. The measuring range of the LS 230 Laser diffraction particle size analyzer is from 0.04 μm to 2000 μm.

According to one particular embodiment of the present invention, 90% of the powder has a diameter of less than 1000 μm, preferably less than 500 μm, and even more preferably less than 400 μm. In particular, 90% of the powder has a diameter of less than 370 μm. This value corresponds to the d₉₀.

According to another particular embodiment of the present invention, 50% of the powder has a diameter of less than 500 μm, preferably less than 300 μm, and even more preferably less than 250 μm. In particular, 50% of the powder has a diameter of less than 220 μm. This value corresponds to the d₅₀.

According to another particular embodiment of the present invention, 10% of the powder has a diameter of less than 300 μm, preferably less than 200 μm, and even more preferably less than 150 μm. In particular, 10% of the powder has a diameter of less than 100 μm. This value corresponds to the d₁₀.

These three values d₉₀, d₅₀ and d₁₀ are also determined by means of the laser diffraction particle size analyzer used for determining the volume mean diameter D4,3.

According to one preferred embodiment of the present invention, the granulated powder consists of pea protein and fibers.

According to another preferred embodiment of the invention, the granulated powder contains pea proteins combined with a mixture of at least two fibers chosen from soluble and insoluble fibers.

Advantageously, the soluble fibers are branched maltodextrins when the insoluble fibers are chosen from leguminous plants fibers and resistant starches, or are a mixture of the two.

According to the invention, the granulated powder contains varying proportions of vegetable proteins and of vegetable fibers.

According to one preferred embodiment, the weight ratio of the vegetable protein, and preferably of the pea protein, to the fibers is between 90:10 and 10:90, preferably between 75:25 and 25:75, more preferably between 65:35 and 35:65. In particular, said ratio is between 60:40 and 45:55, preferably between 55:45 and 45:55.

According to another preferred embodiment, the sum of the amounts of vegetable proteins, and preferably of pea proteins, and of fiber is between 30% and 100%, and preferably between 50% and 100%, of the total mass of said granulated powder (dry/dry).

The applicant company has, to its credit, discovered that, according to these ratios, the functional properties of the powder can be different.

In one embodiment according to the invention, it has been observed, unexpectedly, that, in the food sector, for example, the granulated powder according to the invention has the additional advantage of completely or partially replacing the fats commonly used in recipes.

According to another embodiment of the invention, the granulated powder comprises pea proteins and vegetable fibers, and can also contain any suitable additive, such as flavorings, dyes, stabilizers, excipients, lubricants or preservatives, provided that they do not negatively impact on the desired final functional properties.

These additives may also be pharmaceutical or phytosanitary active ingredients, or detergents. In the present invention, the term “active ingredient” is intended to mean any active molecule which has a pharmacological effect that has been demonstrated and which is of therapeutic interest, also clinically demonstrated.

Said granulated powder in accordance with the invention can also be characterized by its apparent density, determined according to the method of measurement recommended by the European Pharmacopeia (EP 5.1 volume 1, 01/2005: 20915 paragraph 2-9-15; equipment according to FIG. 2-9-15-1).

Under these conditions, said granulated powder advantageously has an apparent density of between 0.30 and 0.90 g/ml, preferably between 0.40 and 0.60 g/ml.

Another functional property of the granulated powder in accordance with the invention is that it has excellent wettability, much better than the wettability noted for the simple mixture. This characteristic is the capacity for water absorption at the surface of a powder. It is proportional to the solubility of the powder and inversely proportional to the formation of lumps. A high wettability makes it possible to confer the “instant” nature on the granulated powder of the present invention.

To measure this wettability, a tall form beaker with a volume of 500 ml is used, and 250 g of distilled water at 20° C.+/−2° C. are placed in said beaker. Exactly 25 g of granulated powder in accordance with the invention or 25 g of the simple mixture are weighed out. At t=0 h, the 25 g of sample are rapidly introduced all at once, and the timer is started. The time necessary for the sample to become completely wet, i.e. for there to be no more sample in dry form, is measured. The test is carried out without stirring and with gentle stirring at 250 rpm. In the test without stirring, the granulated powder in accordance with the present invention becomes wet in less than one minute, preferably in less than 30 seconds, and even more preferably in less than 10 seconds, whereas the simple mixture takes more than one hour to become completely wet.

In the test with gentle stirring, said granulated powder becomes wet in less than 30 s, preferably in less than 10 s, and even more preferably in less than 6 s, whereas the simple mixture takes more than 7 minutes to become completely wet.

In particular, and by way of example, according to the wettability test without stirring described above, a granulated powder composed of pea proteins and of branched maltodextrins becomes wet in less than 10 seconds, very precisely in 8.8 seconds, whereas the simple mixture takes 3 h 45 s to become completely wet. With gentle stirring, the granulated powder becomes wet in 4.3 s, whereas the simple mixture becomes wet in 11 min 20 s.

This test makes it possible to demonstrate that the granulated powder has an “instant” nature compared to the simple mixture which, itself, does not have this “instant” nature.

The granulated powder of the present invention also exhibits a total absence of decantation, i.e. an excellent hold in suspension, which greatly facilitates its use in industrial processes, and represents a major advantage.

The hold in suspension is measured in a 250 ml graduated cylinder. After reconstitution of a 250 ml solution containing 15% of granulated powder according to the invention, in particular by resuspending said powder with gentle stirring, the volume settled out is measured every hour for 7 hours, and then after 24 h and 48 h. There is no decantation of the granulated powder, even after waiting 48 h. This total lack of decantation is not found with the simple mixture. Indeed, one hour after the reconstitution of the mixture, a decantation phenomenon is observed, and accentuates with time.

Other very advantageous technological properties conferred by said granulated powder concern its emulsifying, foaming and gelling capacities, in comparison with the simple mixture of the constituents of this powder.

The emulsifying properties are due to the ability to reduce the interfacial tensions between hydrophilic and hydrophobic components of a food. They are directly related to the solubility of the protein. The powders which have these surface properties will have a considerable potential for use in emulsions in general, in refatted or nonrefatted milk powders, and also in foods containing water and fats (cooked pork meats, meat, condiments).

In the present invention, the emulsifying capacity corresponds to the percentage of “emulsion cream” formed and stable after centrifugation, as a function of the amount of proteins and of the amount of oil. In order to measure it, a 50% rapeseed oil emulsion is prepared, on an ultraturrax at 9500 rpm for one minute, using a solution of granulated powder (hydrated for 10 minutes in demineralized water in order to be free of the ionic forces) at 2%. The emulsion is then centrifuged for 5 minutes at 1500 g. The cream volume is measured in ml. The emulsifying capacity (EC) is calculated using the following formula:

EC (in %)=(cream volume/total volume)×100

The granulated powder exhibits an emulsifying capacity of greater than 50%, preferably greater than 60%, and even more preferably greater than 65%, whereas the simple mixtures have a low emulsifying capacity, less than 20%.

In particular, and by way of example, according to the test for measuring the EC described above, a granulated powder comprising pea proteins and branched maltodextrins has an EC of 68.75%.

Thus, one of the advantageous uses of the granulated powder according to the present invention or capable of being produced according to the implementation of the process for preparing granulated powder according to the invention as described above is its optional use as an emulsifying agent in the compositions mentioned above, for totally replacing any other emulsifying agent, and in particular lecithin. Said granulated powder can itself be totally free of emulsifying agents, considered to be additives according to European regulations. Moreover, one of the advantageous uses of the granulated powder according to the present invention or capable of being produced according to the implementation of the process for preparing granulated powder according to the invention as described below is its optional use as an emulsifying agent in the compositions mentioned above, for totally replacing any other emulsifying agent, and in particular lecithin.

Indeed, the use of said powder makes it possible to completely eliminate lecithin from food formulations, and more particularly food formulations which are totally or partially in the form of an emulsion, i.e. which contain at least two immiscible ingredients (typically water and oil).

In general, emulsifying agents, sometimes called emulsifiers, stabilize emulsions. The emulsifying agents currently used in industries are either purified natural products or synthetic chemical products, the structures of which are very close to those of the natural products.

They are most commonly surfactants or surface agents. They are molecules which possess one end that has an affinity for water (hydrophilic) and one end that has an affinity for oil (hydrophobic). In the food-processing industry, emulsifying agents are used to increase the creaminess of certain products, making it possible to obtain a particular texture. One of the most widely known emulsifying agents is unquestionably lecithin.

Indeed, lecithin, also known as phosphatidylcholine, is conventionally used as an emulsifying agent in the food, cosmetics and other industries. It is a natural emulsifying agent which is made industrially by means of an aqueous treatment of soya oil. It is in the form of a brown-colored pasty liquid. It does not have a very appetizing appearance, nor a very pleasant taste. Lecithin is classified in the lipid category. It can also be extracted from egg yolks, but the process is too expensive to be applied on an industrial scale.

Lecithins are food additives and are subject, like the other food additives, to a strict European regulation which governs the assessment of their innocuousness, their authorization and their labeling. These regulations require that all added emulsifying agents, in whatever form, be mentioned on the packaging of the product, either by virtue of their name or by virtue of their European code (letter E followed by a number, E322 for lecithin) like all the other food additives. What is more, since lecithins are extracted from soya for use on an industrial scale, they have also suffered the repercussions of the negative image conveyed by genetically modified organisms, to which soya can belong.

Thus, the granulated powder according to the present invention or capable of being produced according to the implementation of the process for preparing granulated powder according to the invention as described above, which is preferably itself devoid of emulsifying agents such as lecithin, makes it possible to avoid the use of other emulsifying agents, and in particular of lecithin, and thus makes it possible to be free of both the risks of allergies and the negative image associated with soya, and also the labeling, on the packaging, of lecithin as a food additive.

The foaming properties, which are highly appreciated in patisseries (cakes, souffles, meringues) and in the manufacture of mousses, based on milk or the like, and of whipped creams, are the result of partial unfolding of the proteins which orient themselves at the water/air interface.

In the present invention, the foaming capacity is measured in a 500 ml graduated cylinder. A solution containing 15% of granulated powder in accordance with the present invention is prepared on an Ultraturax at 9500 rpm for 1 minute, before being transferred into the graduated cylinder. The foam volume and the liquid volume are measured every 10 minutes for 30 minutes. The time necessary for the foam to reach 50% of its initial volume is also measured and will make it possible to quantify the stability of the foam.

The granulated powder has an excellent foaming capacity, which is extremely stable over time, whereas the simple mixture foams only very little, and gives a foam which is unstable over time.

Thus, the granulated powder has functional properties (emulsifying capacity, foaming capacity) which have been conferred thereon in particular by the process for preparing said powder.

Another very advantageous property conferred by said granulated powder according to the present invention is the clear improvement in, on the one hand, the taste and, on the other hand, the palatability and the body, which is also defined by the viscosity in the mouth. Indeed the granulated powder has a neutral taste, unlike the simple mixture, which can have a more marked legume taste and consequently curb certain food applications. In some applications, the palatability and the body are also improved compared with the simple mixture.

These very advantageous functional properties which do not exist with a simple mixture mean that they are destined, inter alia, for very diversified and varied applications.

Another aspect of the present invention concerns the use of the granulated powder in the fields of cosmetics, detergence, agrochemistry, industrial and pharmaceutical formulations, construction materials, drilling fluids, in fermentation, in animal feed and in food applications.

Consequently, the present invention also relates to cosmetic, detergent and agrochemical compositions, industrial and pharmaceutical formulations, construction materials, drilling fluids, fermentation media, animal nutritional compositions and food applications comprising the granulated powder according to the present invention or capable of being produced according to the implementation of the process for preparing granulated powder according to the invention as described above.

In these fields, the granulated powder according to the invention may be used in compositions as a functional agent such as an emulsifying, overrun, stabilizing, thickening and/or gelling agent, in particular for totally or partially replacing animal proteins.

Consequently, the present invention also relates to an emulsifying, overrun, stabilizing, thickening and/or gelling agent, which can be used for totally or partially replacing animal proteins, comprising the granulated powder according to the present invention or capable of being produced according to the implementation of the process for preparing granulated powder according to the invention as described above.

One particularly advantageous and valuable use of the present invention relates to the production of preparations intended for clinical nutrition and/or for individuals suffering from undernutrition. This is because the combination of the vegetable proteins, and more particularly pea proteins, with at least one vegetable fiber, and preferably with at least one soluble vegetable fiber, makes it possible to overcome the problem of undernutrition.

Two types of undernutrition exist:
protein undernutrition: protein deficiency predominates, in particular in the case of inflammatory syndrome and protein hypercatabolism. It is also referred to as endogenous undernutrition;
marasmus: there is an energy intake deficit, and protein catabolism is, on the contrary, reduced (adaptation). It is also referred to as exogenous undernutrition, where food intakes are insufficient in terms of amount and of quality.

There are numerous consequences of undernutrition. It is characterized by a loss of energy, most commonly followed by anemia or even asthenia. It also manifests itself through a loss of muscle mass, reducing the physical faculties and increasing the risks of falling. In cases of undernutrition, there is a decrease and an impairment of immune functions. Infections, in particular opportunistic infections, are then more frequent and more serious: Pneumocystis carinii, atypical mycobacteria, mycoses, viral infections (CMV, Herpes, etc.). The risk of mortality increases. Finally, cell renewal is slowed down, and cell tissues suffer because of this.

Elderly individuals are particularly exposed to the problems of undernutrition. However, this is also the case in individuals who live in poor countries, or else individuals who have been hospitalized following serious and long illnesses.

Thus, in cases of undernutrition, the granulated powder according to the present invention or capable of being produced according to the implementation of the process for preparing granulated powder according to the invention as described above can be used in the formulation of high-protein and high-calorie foods intended for oral use.

The granulated powder according to the present invention or capable of being produced according to the implementation of the process for preparing granulated powder according to the invention as described above is used as a total or partial replacement for animal proteins and more particularly milk proteins, customarily used in conventional high-protein cream formulations.

In one preferred embodiment, said foods are in the form of creams, since this food matrix has many advantages in terms of palatability, taste and ease of use. They can be transported easily, can be consumed anywhere, and do not need to be reheated. In addition, they do not need to be chewed and, consequently, do not call for saliva, which is generally lacking in individuals suffering from undernutrition. By virtue of their creamy, homogeneous, smooth and emulsified texture, they can be swallowed directly, with no effort.

Such an application is exemplified in example 4 hereinafter.

Another of the particularly advantageous and valuable uses of the present invention as a total or partial replacement for animal proteins, and more particularly milk proteins, relates to the preparation of a dairy product chosen from the group consisting of fromage frais and ripened cheeses, cheese spreads, fermented milks, milk smoothies, yogurts, specialty dairy products, and ice creams produced from milk.

According to another more preferred embodiment, the powder according to the invention is used for producing cheeses with partial or total replacement of milk proteins.

In the present invention, the term “cheese” denotes a food obtained using coagulated milk or milk products, such as cream, and then optionally draining, possibly followed by a fermentation step and, optionally, by refining (ripened cheeses). The name “cheese” is, according to decree No. 88-1206 of Dec. 30, 1988, reserved for fermented or nonfermented, ripened or nonripened products obtained from materials of exclusively dairy origin (whole milk, partially or totally skimmed milk, cream, fat, buttermilk), used alone or as a mixture, and totally or partially coagulated before draining or after partial elimination of their water.

The milk is acidified, generally using a bacterial culture. An enzyme, rennet, or a substitute such as, for example, acetic acid, vinegar or GDL (glucono-delta-lactone) can then be added in order to cause coagulation and form the curd and the whey.

In the present invention, the term “cheese” also denotes all processed cheeses and all processed cheese spreads. These two types of cheeses are obtained by milling, mixing, melting and emulsification, under the effect of heat and emulsifying agents, of one or more varieties of cheese, with or without the addition of milk constituents and/or other food products (cream, vinegar, spices, enzymes, etc.).

In another preferred embodiment, the powder according to the invention is used for producing yogurts, as total or partial replacement for milk, reconstituted milk powder or milk proteins. Such an application is exemplified in example 3 hereinafter.

Thus, the granulated powder according to the present invention or capable of being produced according to the implementation of the process for preparing granulated powder according to the invention as described above can be used for totally or partially replacing the milk proteins in a food formulation belonging to the group defined by fromage frais and ripened cheeses, processed cheeses or processed cheese spreads, fermented milks, milk smoothies, yogurts, specialty dairy products, and ice creams produced from milk.

The invention thus extends to food formulations comprising a granulated powder according to the invention or capable of being produced according to the implementation of the process for preparing granulated powder according to the invention as described above, or comprising an emulsifying, overrun, stabilizing, thickening and/or gelling agent, which can be used for totally or partially replacing animal proteins, as described above, such as:
beverages,
dairy products (including, for example, fromage frais and ripened cheeses, processed cheeses or processed cheese spreads, fermented milks, milk smoothies, yogurts, specialty dairy products, ice creams produced from milk),
preparations intended for clinical nutrition and/or for individuals suffering from undernutrition,
preparations intended for infant nutrition,
mixtures of powders intended for diet products, or for sportspersons,
soups, sauces and cooking aids,
meat-based products, more particularly in the fine paste and brine sectors, especially in the production of hams,
fish-based products, more particularly surimi-based products,
cereal products such as bread, pasta, cookies, pastries, cereals and bars,
vegetarian products and ready meals.

The granulated powder according to the present invention or capable of being obtained according to the implementation of the process for preparing granulated powder according to the invention as described above also finds applications in animal feed.

The invention will be understood more clearly on reading the examples which follow, which are given as nonlimiting illustrations referring only to certain embodiments and certain advantageous properties according to the invention.

### EXAMPLE 1

### Preparation of a Granulated Powder According to the Invention

A granulated powder containing 60% of pea proteins and 40% of branched maltodextrins was prepared in the following way.

The pea proteins used are sold by the applicant under the name Nutralys® S 85 M. Their total protein content is 85%.

The branched maltodextrins used belong to the Nutriose® range, also sold by the applicant, and are, for example, Nutriose® FB 06.

First of all, a suspension was prepared at a protein/fiber ratio of 60/40 in a stirred tank at a temperature of 50° C.

The mixture has a DM (dry matter content) of 30%.

The mixture obtained was homogenized on a two-stage high-pressure homogenizer (150 bar on the 1st stage and 50 bar on the second) before being dried, in order to have a perfectly homogeneous mixture.

The mixture was spray-dried in a spray-drying tower of MSD type equipped with a high-pressure spray-drying nozzle with recycling of the fine particles at the top of the tower.

The Spray-Drying Conditions are the following:

The injection nozzle was chosen so as to obtain a pressure of 220 bar for a flow rate of 120 l/h.

The air used was at 6 g/kg of moisture.

The air inlet temperatures were set in the following way:
for the inlet air upstream of the top of the tower: temperature of 180° C.,
for the static fluidized bed: temperature of 55° C.,
for the vibrated fluidized bed: temperature of about 20° C.

The outlet temperature was 58° C.

The speed of the air upstream was set at 14.7 m/s and that of the air of the static fluidized bed was 11 m/s.

The granulated powder obtained according to example 1 has the following characteristics:
Moisture content: 5.5%
Dry matter content: 94.5%
Volume mean diameter D4,3: 200 μm.

### EXAMPLE 2

### Measurement of Gelling Capacity

The gelling capacity of the granulated powder obtained according to Example 1 was compared with the gelling capacity of the simple mixture of powder, using the same two constituents: and also the same ratio, as those used to prepare the granulated powder.

1. Solution Preparation

A solution with a concentration of 8% was prepared by placing 8 g of sample (granulated powder or simple mixture of powders) in 100 g of distilled water at 20° C.+/−1° C. 0.3 g of xanthan gum was added to the above solutions in order to avoid decantation of the particles under gravity. The mixture was stirred slowly for 30 min at a speed of 250 rpm in order to allow optimum hydration of the proteins contained in the samples.

2. Measuring Material

The gelatinization of the samples during a heat cycle was characterized, in the oscillatory dynamic mode, by means of the Physica® MCR301 rheometer (Anton Paar) with a striated parallel plate geometry in order to avoid sliding phenomena.

3. Measuring Protocol

1 ml of the hydrated suspension, prepared in paragraph (1), placed between the 50 mm diameter parallel plates, was subjected to a sinusoidal type stress, at the frequency of 1 Hertz and a deformation amplitude of 0.1% to 0.5%, while at the same time applying the following thermal cycle:
1. Heating from 20 to 90° C. in 2000 s-0.5% deformation,
2. Hold at 90° C. for 3600 s-0.2% deformation,
3. Cooling from 90 to 4° C. in 2000 s-0.1% deformation,
4. Hold at 4° C. for 12000 s-0.1% deformation.

4. Interpretation

Monitoring of the storage module G′ and dissipation modulus G″ levels made it possible to characterize the gelling kinetics of the protein under the effect of heat and also the relative level of the force of the gel obtained.

The curves obtained made it possible to measure the gelling speed and the force of the gel obtained, but also the behavior of the gel under cold conditions.

The curves obtained with the granulated powder, in comparison with the curves obtained with the simple mixture, exhibited a faster gelling speed, a higher maximum level, which means that the gels were more solid, and also a better texture and resistance of the gel with respect to cold conditions.

This means that the gelling capacity of the granulated powder was much better than the gelling capacity of the simple physical mixture.

### EXAMPLE 3

### Preparation of Drinkable Yogurts Containing Granulated Powder According to the Present Invention

In this example, the granulated powder was obtained according to the protocol used in Example 1, this time using a pea protein composition/branched maltodextrin weight ratio of 45/55.

The granulated powder therefore contains 45% of a composition of pea proteins (at a total protein content of 85%) and 55% of branched maltodextrins.

The branched maltodextrins used belong to the Nutriose® range, also sold by the applicant, and are, for example, Nutriose® FB 06.

Trials were carried out by replacing the milk with the granulated powder or with the simple mixture of the two constituents. Two replacement percentages were tested: 10% and 50%.

The drinkable yogurts (TRIAL 10 and TRIAL 50) were prepared according to the recipe represented in the table below, and contained the granulated powder of said invention at the two different degrees of replacement. They were then compared with the yogurt containing only milk and also with the control drinkable yogurts (CONTROL 10 and CONTROL 50) prepared in parallel, under the same conditions and not containing the granulated powder according to the present invention, but the simple physical mixture of the two constituents.

The various drinkable yogurts were tasted blind by a trained jury of 20 individuals who were experts in sensory analysis. The following parameters were tested and graded on a scale of 1 to 5, 1 being the poorest grade and 5 the best: color, odor, taste, smoothness in the mouth, consistency, general grade.

1. Recipes Used

| | | Granulated | | “Simple” | |
|---|---|---|---|---|---|
| | | powder | | mixture | |
| | | Degree of replacement | | | |
| | | 10% | 50% | 10% | 50% |
| | CONTROL | T10 | T50 | C10 | C50 |
| Commercially available skimmed | 86 | 77.5 | 43 | 77.5 | 43 |
| milk | | | | | |
| Granulated powder according to | / | 8.5 | 43 | 8.5 | 43 |
| the invention in powder form | | | | | |
| SweetPearl ™ P200 | 10 | 10 | 10 | 10 | 10 |
| Nutriose ® FB06 | 4 | 4 | 4 | 4 | 4 |
| Probiotic ferment BMY-1 | qs | qs | qs | qs | qs |
| Total | 100 | 100 | 100 | 100 | 100 |

SweetPearl™ P200 is the commercial name of maltitol from the applicant company: it is a carbohydrate in crystalline powder form, derived from wheat starch and corn starch.

Nutriose® FB06 is a soluble fiber also sold by the applicant company.

The ferment used is sold by the company CHR Hansen A/S (Denmark).

2. Procedure
The Nutriose® FB06 was dissolved in the milk.
The mixture was then pasteurized at 90° C. for 10 minutes.
This pasteurized mixture was then homogenized using a Niro® Soavi (GEA group) homogenizer, at a pressure of 180 bar.
The resulting emulsion was then cooled to 43° C. and held at this temperature.
The prebiotic ferments were added to the cooled mixture, and the fermentation was checked by continually measuring the pH using a pH-meter.
The fermentation was stopped when the pH of the mixture reached the value of 4.5.
The SweetPearl was then added and the mixture was homogenized with an ALM2 homogenizer, sold by the company Pierre Guerin Technologies (France).
The whole was pasteurized at 90° C. for 15 seconds, in order to eliminate the risks of microbiological contamination.
The whole was cooled to 5° C. before tasting.

3. Results

| | | Granulated | | “Simple” | |
|---|---|---|---|---|---|
| | | powder | | mixture | |
| | | 10% | 50% | 10% | 50% |
| | CONTROL | T10 | T50 | C10 | C50 |
| Color | 5 | 4 | 4 | 3 | 4 |
| Taste | 5 | 5 | 3 | 3 | 2 |
| Smoothness in the mouth | 5 | 4 | 4 | 2 | 2 |
| Consistency | 5 | 5 | 5 | 2 | 1 |
| General grade | 5 | 5 | 4 | 2 | 2 |

This trial demonstrates perfectly that it is entirely possible to replace a part of the milk proteins in a drinkable yogurt with the granulated powder of the present invention, or capable of being produced according to the implementation of the process for preparing granulated powder according to the invention as described above.

The drinkable yogurts containing said granulated powder were judged to be very satisfactory and identical to the control drinkable yogurt containing only milk, with a very slight, but not significant, preference for the drinkable yogurt T10 (degree of replacement of milk proteins 10%). The two drinkable yogurts containing the simple physical mixtures of the two constituents were judged negatively and their assessment demonstrates perfectly that they are not at all similar to the control yogurt, whether in terms of taste, in terms of smoothness in the mouth and in terms of their consistency (judged to be too liquid). Thus, said powder has gelling functional properties which have been conferred upon it in particular by its process of preparation, said properties not being found with the simple mixture of the constituents.

### EXAMPLE 4

### Example of Formulation of High-Protein Cream Containing the Granulated Powder According to the Present Invention

In this example, the granulated powder was obtained according to the protocol used in example 1, this time using a pea protein composition/branched maltodextrin weight ratio of 60/40.

The granulated powder therefore contains 60% of a composition of pea proteins (at a total protein content of 85%) and 40% of branched maltodextrins.

The branched maltodextrins used belong to the Nutriose® range, also sold by the applicant, and are, for example, Nutriose® FB 06.

1. Recipes Used

| | Ingredients |
|---|---|
| | (by weight) |
| Demineralized water | 677.5 |
| Whole milk powder containing 26% | 50.0 |
| fats | |
| Granulated powder according to the | 124.5 |
| invention in powder form | |
| Sugar | 72.3 |
| Clearam ® CR3020 modified starch | 6 |
| Glucidex ® 19IT maltrodextrin | 35.0 |
| Refined rapeseed oil | 26.0 |
| Fontarome ® vanilla flavoring | 8.5 |
| Acesulfame K | 0.2 |
| TOTAL | 1000 |

2. Procedure
The demineralized water was heated to 70° C. Some of this water was used to dissolve the vanilla flavoring.
The various powders (milk, granulated powder, sugar, modified starch, maltodextrin) were mixed dry, before being added to the hot water. The whole was mixed and kept at 70° C. by heating.
The rapeseed oil was then added to the above mixture.
The whole was mixed using a high-capacity mixer (Polytron PT 45/80, Kinematica, Switzerland) for 4 minutes on speed 4.
The flavoring was then added and the whole was pasteurized at 130° C. for 175 seconds, in order to eliminate the risks of microbiological contamination.
The whole was cooled to ambient temperature before tasting.

3. Results

The nutritional characteristics of the high-protein cream prepared with the granulated powder according to the present invention are given in the table below.

| | Value in g. 100 g |
|---|---|
| | of cream prepared |
| Energy value | 147 = 165 Kcal/100 ml |
| Protein content | 9.2 |
| Carbohydrate content | 13.7 |
| Sugar content | 9.7 |
| Fat content | 3.96 |
| Fiber content | 2.55 |
| Water content | 69 |

In terms of energy value, the cream provides 165 Kcal per 100 ml. This means that, by consuming 500 ml of cream, the individual already ingests approximately 825 Kcal, i.e. ⅓ of said individual's daily needs (on the basis of a need of 2400 Kcal/day for a man).

In terms of total energy intake, the proteins contained in the cream contribute in an amount of 29% to this intake, the carbohydrates in an amount of 43% and the fats in an amount of 28%.

The cream was tasted by a trained jury of 20 individuals who were experts in sensory analysis, and was compared with a commercially available cream having the same purpose, but containing only milk proteins. It was judged to be entirely satisfactory and identical in every way to the commercially available cream, both in terms of consistency in the mouth and in terms of smoothness and taste.

It was judged to be more easily digestible owing to its greatly reduced content of milk proteins which can sometimes be sickly, especially in individuals who are ill.

1-26. (canceled)

27. A granulated powder comprising at least one vegetable protein and at least one vegetable fiber, wherein said granulated powder has:
a laser volume mean diameter D4,3 of between 10 μm and 500 μm, and
a dry matter content, determined after stoving at 130° C. for 2 hours, of greater than 80%.

28. The granulated powder according to claim 27, wherein the weight ratio of the vegetable protein to the vegetable fiber is between 99:1 and 1:99.

29. The granulated powder according to claim 27, wherein the sum of the amounts of vegetable protein and of vegetable fiber is between 30% and 100% of the total mass of said granulated powder (dry/dry).

30. The granulated powder according to claim 27, wherein the vegetable fiber is selected from the group consisting of soluble fiber, insoluble fiber and mixtures thereof.

31. The granulated powder according to claim 30, wherein said soluble vegetable fiber is selected from the group consisting of fructans, glucooligosaccharides (GOSs), isomaltooligosaccharides (IMOs), trans-galactooligosaccharides (TOSs), polydextrins, polydextrose, branched maltodextrins, indigestible dextrins and soluble oligosaccharides derived from oleaginous plants or protein-producing plants.

32. The granulated powder according to claim 30, wherein said insoluble vegetable fiber is selected from the group consisting of resistant starches, cereal fiber, fruit fiber, fiber from vegetables, leguminous plants fiber and mixtures thereof.

33. The granulated powder according to claim 27, wherein the vegetable protein is a protein derived from cereal plants, oleaginous plants, leguminous plants, tuberous plants, or algae and microalgae, used alone or as a mixture and chosen from the same family or from different families.

34. The granulated powder according to claim 33, wherein the vegetable protein is a leguminous plant protein, said leguminous plant being selected from the group consisting of alfalfa, clover, lupine, pea, bean, broad bean, horse bean and lentil, and mixtures thereof.

35. The granulated powder according to claim 34, wherein said granulated powder comprises proteins and fibers derived from a leguminous plant selected from the group consisting of alfalfa, clover, lupine, pea, bean, broad bean, horse bean and lentil, and mixtures thereof.

36. The granulated powder according to claim 34, wherein said leguminous plant protein is pea.

37. The granulated powder according to claim 27, wherein said granulated powder comprises pea proteins and a soluble vegetable fiber.

38. The granulated powder according to claim 27, wherein said granulated powder comprises pea proteins and an insoluble vegetable fiber.

39. The granulated powder according to claim 27, wherein said granulated powder does not contain gluten.

40. The granulated powder according to claim 36, wherein said granulated powder does not comprise any emulsifying agent other than the pea proteins, and, optionally, does not comprise any lecithin or derivatives thereof

41. The granulated powder according to claim 27, wherein said granulated powder exhibits:
an apparent density of between 0.30 and 0.90 g/ml;
a wettability of less than 30 s;
a total absence of decantation; and
an emulsifying capacity of greater than 50%.

42. A process for manufacturing a granulated powder according to claim 27, comprising conjointly drying at least two constituents and bringing at least one vegetable protein into intimate contact with at least one vegetable fiber wherein said step of bringing into intimate contact results in a final dry matter content, determined after stoving at 130° C. for 2 hours, of greater than 80%.

43. The process for manufacturing a granulated powder according to claim 42, wherein the vegetable protein is a pea protein and the vegetable fiber is a soluble fiber.

44. The process for manufacturing a granulated powder according to claim 42, wherein the process comprises a step of spray-drying a suspension of at least one vegetable protein and of at least one vegetable fiber, said spray-drying step being followed by a step of granulation of the spray-dried powder.

45. The process for manufacturing a granulated powder according to claim 42, wherein said process comprises:
a) preparing, at a temperature between 15 and 70° C., a suspension of pea proteins and of branched maltodextrins, in which:
said pea proteins have a soluble protein content of between 20% and 99%;
said branched maltodextrins have between 15% and 35% of 1-6 glucosidic linkages, a reducing sugar content of less than 20%, a molecular weight (MW) of between 4000 and 6000 g/mol and a number-average molecular mass (Mn) of between 250 and 4000 g/mol;
the weight ratio of the pea proteins to the branched maltodextrins is between 99:1 and 1:99;
the dry matter content of the suspension is between 25% and 50%;
b) carrying out an optional step of heat treatment at high temperature and for a short period of time in order to reduce bacteriological risks;
c) carrying out an optional step of high-pressure homogenization of the suspension obtained according to a), and independently of optional step b);
d) maintaining said suspension of pea proteins and of branched maltodextrins at a temperature of between 15 and 80° C., or, in the event of step b) being carried out, bringing said suspension of pea proteins and of branched maltodextrins back to a temperature of between 15 and 80° C.;
e) spray-drying said suspension in a spray-drying tower equipped with a high-pressure spray-drying nozzle with recycling of the fine particles at the top of the tower;
f) granulating in said spray-drying tower; and
g) recovering the resulting granulated powder comprising the pea proteins and the branched maltodextrins.

46. A cosmetic, detergent or agrochemical composition, industrial or pharmaceutical formulations, construction materials, drilling fluids, a fermentation medium, an animal nutritional composition or food applications comprising the granulated powder according to claim 27.

47. An emulsifying, overrun, stabilizing, thickening and/or gelling agent comprising the granulated powder according to claim 27.

48. A food formulation selected from the group consisting of beverages, dairy products, preparations intended for clinical nutrition and/or for individuals suffering from undernutrition, preparations intended for infant nutrition, mixtures of powders intended for diet products or for sportspersons, soups, sauces and cooking aids, meat-based products, fish-based products, all types of confectionary, cereal products, vegetarian products and ready meals, comprising an emulsifying, stabilizing, thickening and/or gelling agent according to claim 47.

49. The food formulation according to claim 48, wherein the dairy product is selected from the group consisting of fromage frais, ripened cheeses, processed cheeses, processed cheese spreads, fermented milks, milk smoothies, yogurts, specialty dairy products, and ice creams produced from milk.
